(19) United States
(12) Patent Application Publication
(10) Pub. No.: US 2023/0086701 A1
(43) Pub. Date: Mar. 23, 2023

(54) SYSTEM FOR CUMULATIVE IMAGING OF BIOLOGICAL SAMPLES

(71) Applicant: Bio-Rad Laboratories, Inc., Hercules, CA (US)

(72) Inventors: Evan THRUSH, San Anselmo, CA (US); Stephen SWIHART, Walnut Creek, CA (US); Kevin McDONALD, Novato, CA (US)

(21) Appl. No.: 17/946,555

(22) Filed: Sep. 16, 2022

Related U.S. Application Data
(60) Provisional application No. 63/281,993, filed on Sep. 17, 2021.

(51) Int. Cl. G01N 21/64; G06T 5/50; G06V 10/60; G01N 21/76; G01N 33/68

(52) U.S. Cl. CPC G01N 21/6486; G01N 21/76; G01N 33/68; G06T 5/50; G06V 10/60; G06T 2207/10016; G06T 2207/10144; G06T 2207/20221

(57) ABSTRACT

Aspects of the present disclosure relate to systems and methods for generating a composite image. This can include a western blot imager with a real time camera. One aspect of the present disclosure relates to an imaging system. The imaging system includes a sample plane that can receive and hold a sample, a photon resolving camera, and a lens attached to the photon resolving camera, the photon resolving camera and the lens positioned to image the sample plane.

### CROSS REFERENCE TO RELATED PATENT APPLICATIONS

The present application claims benefit of priority to U.S. Provisional Pat. Application No. 63/281,993, filed Sep. 17, 2021, which is incorporated by reference for all purposes.

### BACKGROUND OF THE INVENTION

Imaging can be used in the evaluation and/or monitoring of a biological process. This imaging can include luminescence imaging, and specifically fluorescence and/or chemiluminescence imaging. Imaging can produce images via a variety of techniques such as microscopy, imaging probes, and spectroscopy. Imaging can include blotting, such as a western blot. Western blotting can be used to detect specific biological material in a sample, such as, specific proteins.

### BRIEF SUMMARY

One aspect of the present disclosure relates to an imaging system. The imaging system includes a sample plane that can receive and hold a sample, a photon resolving camera, and a lens attached to the photon resolving camera, the photon resolving camera and the lens positioned to image the sample plane.

In some embodiments, the imaging further includes a processor. In some embodiments, the photon resolving camera and the processor can perform fluorescent and/or chemiluminescent imaging of a biological sample. In some embodiments, the photon resolving camera and the processor can image of a western blot sample.

In some embodiments, the sample can be a fluorescent and/or chemiluminescent biological sample. In some embodiments, the sample can be a western blot sample. In some embodiments, the processor can generate a series of images of the sample plane. In some embodiments, each of the series of images can have the same exposure time. In some embodiments, at least some of the images in the series of images have different exposure times.

In some embodiments, the processor can generate a composite image from selection of images in the series of images. In some embodiments, the processor can generate and provide a live image stream displaying the composite image updated as a new image in the series of images is generated.

One aspect of the present disclosure relates to a method of fluorescent and/or chemiluminescent imaging of a biological sample. The method includes generating a series of images of the biological sample with a photon resolving camera, generating a composite image from at least some of the series of images, and providing the composite image to a user.

In some embodiments, the method includes providing the series of images to a user, and receiving an input selecting at least some of the images in the series of images. In some embodiments, the composite image is generated from the selected at least some of the images in the series of images. In some embodiments, generating a series of images includes setting an exposure time, and capturing images at the set exposure time.

In some embodiments, the method includes identifying a brightness level of at least one pixel of one of the images, modifying the exposure time based on the brightness level to achieve a desired brightness level in a next captured image, and capturing a next image at the modified exposure time. In some embodiments, the at least one pixel can be the brightest pixel in the image. In some embodiments, modifying the exposure time to achieve a desired brightness level includes increasing the exposure time to increase the brightness level of the brightest pixel in the image.

In some embodiments, the at least one pixel can be the brightest pixel in the image. In some embodiments, modifying the exposure time to achieve a desired brightness level includes decreasing the exposure time from a first exposure time to a second exposure time to decrease the brightness level of the brightest pixel in the image.

In some embodiments, the exposure time is set to a first exposure time. In some embodiments, the method includes identifying at least one pixel as saturated, modifying the exposure time from the first exposure time to a second exposure time to decrease a brightness level of the saturated at least one pixel, capturing image data at the modified exposure time of the at least one pixel, determining that the at least one pixel is not saturated, scaling the at least one pixel based on the second exposure time, and replacing the saturated at least one pixel with the scaled at least one pixel.

In some embodiments, modifying the exposure time from the first exposure time to the second exposure time includes decreasing the exposure time such that the second exposure time is less than the first exposure time. In some embodiments the at least one pixel is scaled based on both the first exposure time and the second exposure time.

In some embodiments, generating the composite image includes receiving a first input selecting a first set of images and a first portion of each of the images in the first set of images, receiving a second input selecting a second set of images and a second portion of each of the images in the second set of images, generating a first composite portion from the first portion of each of the images in the first set of images, generating a second composite portion from the second portion of each of the images in the second set of images, and combining the first composite portion and the second composite portion.

### BRIEF DESCRIPTION OF THE DRAWINGS

FIG. 1 is a schematic illustration of one embodiment of an imaging system.

FIG. 2 is a schematic illustration of one embodiment of a computer for use with an imaging system.

FIG. 3 is a flowchart illustrating one embodiment of a process for imaging of a biological sample.

FIG. 4 is a flowchart illustrating one embodiment of a process for an aspect of generating a series of images of a biological sample.

FIG. 5 is a flowchart illustrating one embodiment of a process for another aspect of generating a series of images of a biological sample.

FIG. 6 is a flowchart illustrating one embodiment of a process for generating a composite image of a biological sample.

### DETAILED DESCRIPTION

Imaging of biological samples presents challenges due to the wide range of luminescence from different portions of a sample. In fact, it can occur that the range of luminescence in a sample is greater than the dynamic range of the cameras and/or sensors used in generating the image data. When this occurs, the camera and/or sensor can be set to a single set of exposure parameters, which can, in some embodiments, sacrifice performance at either the high or low range of luminescence in the sample. This can degrade image quality and result in complicated post-processing to enable analysis of the sample.

Systems and methods disclosed herein address these challenges via the use of a photon resolving camera. Such a camera can enable unique operation of the imaging system. Due to low read noise, which can enable each pixel to count photons, significantly shorter exposure times can be used. This can decrease the likelihood of saturation of pixels during the generation of image data. With this shorter exposure time, a series of images can be generated. These images can have the same exposure time or can have different exposure times.

All or portions of some or all of the images in the series of images can be combined to generate a composite image. Via the generation of the composite image, signals from individual images are additive. Thus, weak signals at the pixel level can be strengthened via the generation of the composite image. Cameras which are not capable of photon counting are not practical for additive imaging of high numbers of short integration time, low intensity images. At the lowest intensity levels, the individual pixel values can be from either a photon event or from random electronic noise in the readout electronics. Therefore, a photon event is indistinguishable from variation in readout values. In a photon counting camera the bias voltage from a pixel is consistent between readouts so an increase in voltage above bias is known to be a photon event, and therefore, appropriate to be used in additive data accumulation over many images.

Further, this aggregation can, in some embodiments, occur in real time via providing a streamed image to a user. This streamed image can, at a given instant, show the composite image including all captured images. As new images are captured, the composite image shown in the streamed image can be updated. Thus, the user can see the composite image as it is being generated from the growing series of images.

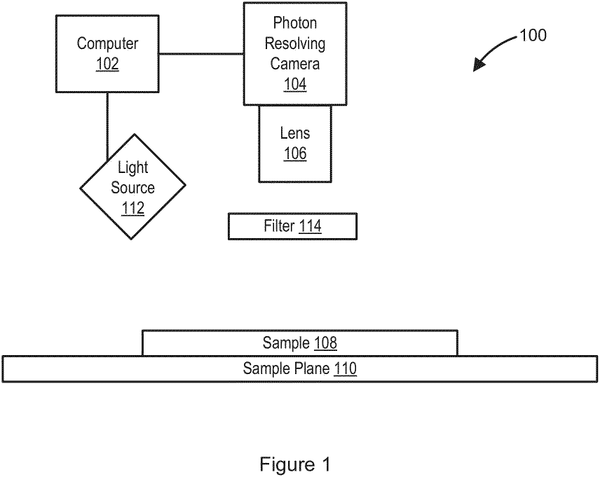

With reference now to FIG. 1, a schematic illustration of one embodiment of an imaging system 100 is shown. The imaging system 100 can be configured for imaging of a biological sample, and specifically can be configured for fluorescent and/or chemiluminescent imaging of a biological sample. In some embodiments, this can include the imaging system 100 being configured for imaging of a western blot sample.

The imaging system 100 can include a computer 102. The computer 102 can be communicatingly coupled with one or several other components of the imaging system 100, and can be configured to receive information from these one or several other components and to generate and/or send one or several control signals to these one or several other components of the imaging system 100. The processor 100 can operate according to stored instructions, and specifically can execute stored instructions in the form of code to gather information from the one or several components of the imaging system 100 and/or to generate and/or send one or several control signals to the one or several other components of the imaging system.

The computer 102 can be communicatingly coupled with a photon resolving camera 104. In some embodiments, the computer 102 can receive information such as image data from the photon resolving camera 104 and can control the photon resolving camera 104 to generate image data, and specifically to generate a series of image of a sample on a sample plane. In some embodiments, this can include setting one or several parameters of the photon resolving camera 104 such as, for example the exposure time. In some embodiments, the computer 102 can control the photon resolving camera 104 such that each of the images in the series of images has the same exposure time, and in some embodiments, the computer 102 can control the photon resolving camera 104 such that some of the images in the series of images have different exposure times. In some embodiments, the computer 102 can generate control signals directing the photon resolving camera 104 to gather image data from all pixels in the photon resolving camera 104 and/or from a subset of all pixels in the photon resolving camera 104.

In some embodiments, the computer 102 can receive the image data from the photon resolving camera 104, which image data can comprise a plurality of images generated at different times. In some embodiments, this image data can comprises a series of images, which can be sequentially generated by the photon resolving camera 104 according to one or several control signals received from the computer 102. The computer can provide all or portions of the series of images to the user and can, in some embodiments, generate a composite image from some or all of the images in the series of images. In some embodiments, the computer 102 can generate a composite image from portions of a plurality of subsets of images in the series of images.

In some embodiments, the computer 102 can receive image data from the photon resolving camera 104, which image data can comprise a series of images. As each of the series of images is generated by the photon resolving camera 104, the image can be provided to the computer 102. The computer 102 can, in some embodiments, generate a composite image from the images received from the photon resolving camera 104, thereby creating a streamed image. Thus, in some embodiments, when the computer 102 receives an image from the photon resolving camera 104, the computer 102 can add the received image to a previously received image to generate a composite image. If a composite image has been previously generated for the sample being imaged, the computer 102 can add the received image to the previously generated composite image and/or to the previously received images to generate an updated composite image. This updated composite image can, in some embodiments, be provided to the user, and can continue to be updated as further images are received from the photon resolving camera 104. Thus, the computer 102 can be configured to generate a provide an image stream displaying the composite image updated as new images, and in some embodiments, as each new image, in the series of images is generated.

In some embodiments, each pixel of the photon resolving camera can count photons. In some embodiments, the photon resolving camera can have low read noise such as, for example, less than 0.3 electrons rms. Due to the low read noise, multiple images in the series of images can be combined to generate a composite image, and specifically, multiple images having relatively short exposure times can be combined to generate the composite image. In some embodiments, these exposure times, and specifically, these relatively short exposure times can include exposure times from, for example, 0.1 seconds to 30 seconds, 0.3 seconds to 20 seconds, 0.5 seconds to 10 seconds, or the like.

The camera 104 can be coupled with a lens 106. In some embodiments, the lens 106 can comprise a high numerical aperture lens. The lens 106 can be configured to enable imaging by the camera 104 of a sample 108 that can be located on a sample plane 110. The sample 108 can comprise a biological sample, and specifically can comprise a blot sample such as, for example, a western blot sample. In some embodiments, the sample can comprise a fluorescent and/or chemiluminescent biological sample. The sample plane 110 can comprise an area for holding the sample 108. In some embodiments, the sample plane 110 can comprise a planar area with one or several features configured to secure the sample 108 in a desired position.

In some embodiments, the imaging system 100 can further include a light source 112. The light source 112 can be configured to illuminate all or portions of the sample plane 110 and all or portions of the sample 108. In some embodiments, the light source 112 can enable fluorescence imaging and can comprise a source of excitation energy. In some embodiments, and as depicted in FIG. 1, the light source 112 can be communicatingly coupled with the computer 102 such that the computer 102 can control the operation of the light source 112, and specifically can control the light source 112 to illuminate the sample 108 at one or several desired times and in a desired manner.

The imaging system can further include one or several filters 114. Some or all of the one or several filters 114 can comprise an emission filter, and can be configured to filter out electromagnetic radiation within an excitation range, and specifically can filter out excitation energy from the light source 112. In some embodiments, the filter can transmit emission energy being emitted by one or several fluorophores in the sample 108. Some or all of the one or several filters 114 can be placed in different locations. In some embodiments, and as shown in FIG. 1, some or all of the filters 114 can be placed before the lens 106 to be positioned between the lens 106 and the sample 108 and/or sample plane 110. In some embodiments, some or all of the filters 114 can be placed behind the lens 106 to be positioned between the lens 105 and the photon resolving camera 104. In some embodiments, and when some or all of the filters 114 comprise an emission filter configured to filter out undesired electromagnetic radiation from the excitation light source, these some or all of the filters 114 can be placed in front of the light source 112 to be positioned between the light source 112 and the sample 108 and/or the sample plane 110.

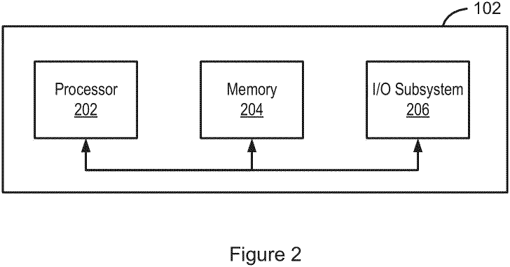

With reference now to FIG. 2, a schematic illustration of one embodiment of the computer 102 is shown. The computer 102 can comprise one or several processors 202, memory 204, and an input/output (“I/O”) subsystem 206.

The processor 202, which may be implemented as one or more integrated circuits (e.g., a conventional microprocessor or microcontroller), controls the operation of the computer 102 and the imaging system 100. One or more processors, including single core and/or multicore processors, may be included in the processor 202. Processor 202 may be implemented as one or more independent processing units with single or multicore processors and processor caches included in each processing unit. In other embodiments, processor 202 may also be implemented as a quad-core processing unit or larger multicore designs (e.g., hexa-core processors, octo-core processors, ten-core processors, or greater.

Processor 202 may execute a variety of software processes embodied in program code, and may maintain multiple concurrently executing programs or processes. At any given time, some or all of the program code to be executed can be resident in processor(s) 202 and/or in memory 204. In some embodiments, computer 102 may include one or more specialized processors, such as digital signal processors (DSPs), outboard processors, graphics processors, application-specific processors, and/or the like.

The computer 102 may comprise memory 204, comprising hardware and software components used for storing data and program instructions, such as system memory and computer-readable storage media. The system memory and/or computer-readable storage media may store program instructions that are loadable and executable on processor 202, as well as data generated during the execution of these programs.

Depending on the configuration and type of computer 102, system memory may be stored in volatile memory (such as random access memory (RAM)) and/or in non-volatile storage drives (such as read-only memory (ROM), flash memory, etc.). The RAM may contain data and/or program modules that are immediately accessible to and/or presently being operated and executed by processor 202. In some implementations, system memory may include multiple different types of memory, such as static random access memory (SRAM) or dynamic random access memory (DRAM). In some implementations, a basic input/output system (BIOS), containing the basic routines that help to transfer information between elements within computer 102, such as during start-up, may typically be stored in the non-volatile storage drives. By way of example, and not limitation, system memory may include application programs, such as client applications, Web browsers, mid-tier applications, server applications, etc., program data, and an operating system.

Memory 204 also may provide one or more tangible computer-readable storage media for storing the basic programming and data constructs that provide the functionality of some embodiments. Software (programs, code modules, instructions) that when executed by a processor provide the functionality described herein may be stored in memory 204. These software modules or instructions may be executed by processor 202. Memory 204 may also provide a repository for storing data used in accordance with the present invention.

Memory 204 may also include a computer-readable storage media reader that can further be connected to computer-readable storage media. Together and, optionally, in combination with system memory, computer-readable storage media may comprehensively represent remote, local, fixed, and/or removable storage devices plus storage media for temporarily and/or more permanently containing, storing, transmitting, and retrieving computer-readable information.

Computer-readable storage media containing program code, or portions of program code, may include any appropriate media known or used in the art, including storage media and communication media, such as, but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media. This can also include nontangible computer-readable media, such as data signals, data transmissions, or any other medium which can be used to transmit the desired information and which can be accessed by computer 102.

By way of example, computer-readable storage media may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media may include, but is not limited to, Zip® drives, flash memory cards, universal serial bus (USB) flash drives, secure digital (SD) cards, DVD disks, digital video tape, and the like. Computer-readable storage media may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer 102.

The input/output module 206 (I/O module 206 or I/O subsystem 206) can be configured to receive inputs from the user of the imaging system 100 and to provide outputs to the user of the imaging system 100. In some embodiments, the I/O subsystem 206 may include device controllers for one or more user interface input devices and/or user interface output devices. User interface input and output devices may be integral with the computer 102 (e.g., integrated audio/video systems, and/or touchscreen displays). The I/O subsystem 206 may provide one or several outputs to a user by converting one or several electrical signals to user perceptible and/or interpretable form, and may receive one or several inputs from the user by generating one or several electrical signals based on one or several user-caused interactions with the I/O subsystem 206 such as the depressing of a key or button, the moving of a mouse, the interaction with a touchscreen or trackpad, the interaction of a sound wave with a microphone, or the like.

Input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. Input devices may also include three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additional input devices may include, for example, motion sensing and/or gesture recognition devices that enable users to control and interact with an input device through a natural user interface using gestures and spoken commands, eye gesture recognition devices that detect eye activity from users and transform the eye gestures as input into an input device, voice recognition sensing devices that enable users to interact with voice recognition systems through voice commands, medical imaging input devices, MIDI keyboards, digital musical instruments, and the like.

Output devices may include one or more display subsystems, indicator lights, or non-visual displays such as audio output devices, etc. Display subsystems may include, for example, cathode ray tube (CRT) displays, flat-panel devices, such as those using a liquid crystal display (LCD) or plasma display, light-emitting diode (LED) displays, projection devices, touch screens, and the like. In general, use of the term “output device” is intended to include all possible types of devices and mechanisms for outputting information from the computer 102 to a user or other computer. For example, output devices may include, without limitation, a variety of display devices that visually convey text, graphics, and audio/video information such as monitors, printers, speakers, headphones, automotive navigation systems, plotters, voice output devices, and modems.

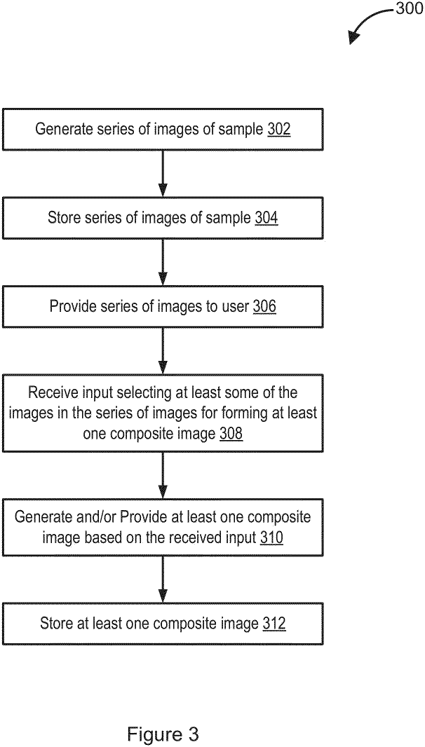

With reference now to FIG. 3, a flowchart illustrating one embodiment of a process 300 for imaging of a biological sample is shown. The process 300 can be performed by all or portions of the imaging system 100. The process 100 begins at block 302, wherein a series of images of a sample are generated. In some embodiments, this can include the computer 102 directing the photon resolving camera 104 to generate a series of images, and specifically to repeatedly capture image data of the same sample at different times. In some embodiments, the computer 102 can generate and send control signal directing the photon resolving camera 104 to generate the series of images, and controlling the operation of the photon resolving camera 104 in generating the series of images. The computer 102 can, for example, direct generation of a desired number of images, generation of images for a desired duration of time, generation of images at a desired frequency, or the like. In some embodiments, the computer 102 can direct the photon resolving camera 104 to operate according to one or several parameters, including, for example, setting an exposure time for generation of the image data.

In some embodiments, and as a part of generating the series of images, the computer 102 can generate and send one or several control signal directing the operation of the light source. In some embodiments, this can include controlling: an intensity of illumination generated by the light source 112; one or several frequencies of illumination generated by the light source 112; a timing and/or duration of illumination generated by the light source 112; and/or portions of the sample 108 and/or sample plane 110 to be illuminated.

In response to receipt of the control signals, the light source 112 can generate directed illumination, and the photon resolving camera 104 can generate a series of images. This can include, for example, generating a directed number of images, generating images for a directed period of time, generating images at a desired frequency, generating images having a set exposure time, or the like. In some embodiments, for example, the computer 102 can set an exposure time based on one or several user inputs, can provide instructions to the photon resolving camera 104 to generate images at the set exposure time, and the photon resolving camera 104 can capture images in the series of images at the set exposure time. The photon resolving camera 104 can send the generated images to the computer 102.

At block 304, the computer receives the generated series of images, and stores the series of images of the sample. In some embodiments, this can include the storing of the series of images in the memory 204, and specifically in one or several databases in the memory 204.

At block 306, all or portions of the series of images is provided to the user. In some embodiments, the all or portions of the series of images can be provided to the user via the computer 102 and specifically via the I/O subsystem 206. In some embodiments, the images can be presented to the user in the form of a streamed image while the series of images is being generated, and/or in some embodiments, the all or portions of the series of images can be presented to the user after the completion of the generation of the series of images. In some embodiments, the streamed image can be generated by continuously summing the generated images, such that each newly generated image is added to a composite image formed by the combination of some or all of the previously generated images. This adding of the newly generated image to the previously formed composite image can create a new composite image. The new composite image can be provided to the user and/or displayed to the user via the I/O subsystem 206.

In some embodiments, the generation of the image stream composite image can result in a composite image at the start of the generation of the series of images that is faint, but that becomes less faint as each newly generated image is added. By the end of the generation of the series of images, this composite image can be relatively brighter than the composite image at the start of the generation of the series of images.

In some embodiments, the user can leave the streamed image and can view one or several composite images formed from the combination of previously captured images in the series of images. In some embodiments, the user can scroll through frames of the composite image, each frame representing a different number of combined images forming the composite images. In some embodiments, scrolling through frames of the composite image in a first direction can decrease the number of images combined in the composite image, and in some embodiments, scrolling in a second direction, which second direction can be opposite to the first direction, can increase the number of images combined in the composite image. In some embodiments, this first direction can correspond to moving earlier in the timeframe in which the series of images was generated and thereby moving to a frame in which the composite image is formed by a smaller number of images. In some embodiments, this second direction can correspond to moving later in the timeframe in which the series of images was generated and thereby moving to a frame in which the composite image is formed by a larger number of images.

At block 308, an input selecting at least some of the images in the series of images is received. This input can direct the forming of at least one composite image and can identify one or several images in the series of images for inclusion in the composite image. In some embodiments, this input can be received in response to the series of images provided to the user in block 306. In some embodiments, for example, the user can select one or several images of the series of images for inclusion in the composite image and/or the user can select one or several portion of one or several images for inclusion in the composite image. The user can provide these inputs via the I/O subsystem 206.

At block 310, a composite image is generated and/or provided to the user. The composite image can be generated by the computer 102 based on the input received in block 308, and specifically can be generated from at least some of the series of images. In some embodiments, the composite image can be generated from the selected at least some of the images in the series of images. In some embodiments, the composite image can be generated by the adding together of selected images from the series of images and/or by adding together the one or several portions of images selected from the series of images. After the composite image has been generated, the composite image can be provided to the user via the I/O subsystem 206 of the computer 102.

At block 312, the composite image is stored. In some embodiments, the composite image can be stored in the memory 204, and specifically in a database of composite images in the memory.

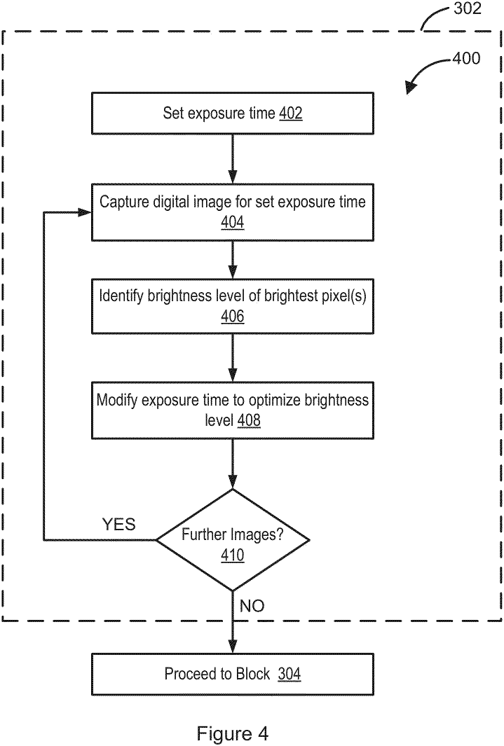

With reference now to FIG. 4, a flowchart illustrating one embodiment of a process 400 for an aspect of generating a series of images of a biological sample is shown. The process 400 can be performed as a part of, or in the place of the step of block 302 of FIG. 3. The process 400 begins at block 402, wherein an exposure time is set. In some embodiments, this exposure time can be a first exposure time. In some embodiments, the exposure time can be set by the computer 102 based on one or several inputs received from the user. In some embodiments, the exposure time can be set by the computer 102 based on one or several rules and/or based on one or several stored default exposure times. In some embodiments, the first exposure time can be set to a time selected to decrease a likelihood of saturation of pixels in the image. In some embodiments, for example, in the range of potential exposure times, the first exposure time can be set to an exposure time shorter than 50 percent of potential exposure times, shorter than 75 percent of exposure times, shorter than 90 percent of exposure times, or the like. In some embodiments, and as part of setting the exposure time, the computer 102 send one or several control signals specifying the exposure time to the photon resolving camera 104. The photon resolving camera 104 can receive these control signals and can be set to generate image according to the exposure time.

At block 404, the photon resolving camera 104 captures one or several digital images for the set exposure time. The digital image is evaluated, and as indicated in block 406, a brightness level of one or several brightest pixels is identified. As used herein, a brightness level can correspond to a signal relative to a maximum value. For example, pixels in imaging sensors can saturate, at which point they cannot sense any further increase in photon exposure. In some embodiments, the one or several brightest pixels can be the one or several pixels sharing a common brightness level which is the highest of all brightness levels of pixels in the digital image. In some embodiments, the one or several brightest pixels can be the one or several pixels comprising a portion of pixels having highest brightness levels of all brightness levels of pixels in the digital image. This can include, for example, the highest 1% of brightness levels, the highest 2% of brightness levels, the highest 3% of brightness levels, the highest 5% of brightness levels, the highest 10% of brightness levels, or the like. In some embodiments, the one or several brightest pixels can be identified by the computer 102, and the brightness levels of these one or several brightest pixels can be identified by the computer 102.

At block 408, the computer 102 modifies the set exposure time to achieve a desired brightness level in a next captured. In some embodiments, the computer 102 modifies the set exposure time to optimize pixel brightness. In some embodiments this optimized level can, for example, correspond to a desire level within a dynamic range of one or several pixels. In some embodiments, a brightness level that optimizes pixel brightness can include a brightness level that achieves a desired percent of saturation of, for example, one or several pixels, one or several capacitors storing accumulated charge for a pixel, an analog-to-digital converter, or the like.

The exposure time can be modified based on, for example, the brightness level of the one or several brightest pixels. For example, if the one or several brightest pixels have too low a brightness level, then the computer 102 can increase the exposure time to increase the brightness level of pixels, including the one or several brightest pixels, in the next generated image. Alternatively, if the brightest pixels have too high a brightness level, or in some embodiments, are fully saturated, then the computer 102 can decrease the exposure time to decrease the brightness level of pixels, including the one or several brightest pixels, in the next generated image. In some embodiments, the computer 102 can increase the exposure time based on the brightness level of the one or several brightest pixels. For example, if the one or several brightest pixels are at 50% of maximum brightness, then the computer 102 may double the exposure time, whereas if the one or several brightest pixels are at 80% maximum brightness, then the computer 102 may increase the exposure time by 25%. If the one or several brightest pixels are at a maximum brightness level and/or are saturated, then the computer 102 can decrease the exposure time by, for example, a predetermined value such as, for example, 50%, 25%, 10%, 5%, between 0 and 50%, or any other or intermediate percent. The computer 102 can generate one or several control signals and can send these one or several control signals to the photon resolving camera 104 modifying the exposure time. The photon resolving camera 104 can receive this signal and can modify the exposure time of the next generated image.

At decision step 410, it is determined if further images should be captured. This can include determining if the entire series of images has already been captured and/or generated, or alternatively if additional images are desired in the series of image. In some embodiments, the computer 102 can track the number of images generated in the series of images and/or the duration of time during which images in the series of images have been captured, and based on this information can determine if further images should be captured. If it is determined that further images are to be captured, then the process 400 returns to block 404 and proceed as outlined above. Alternatively, if it is determined that no further images are to be captured, then the process 400 proceeds to block 304 of FIG. 3.

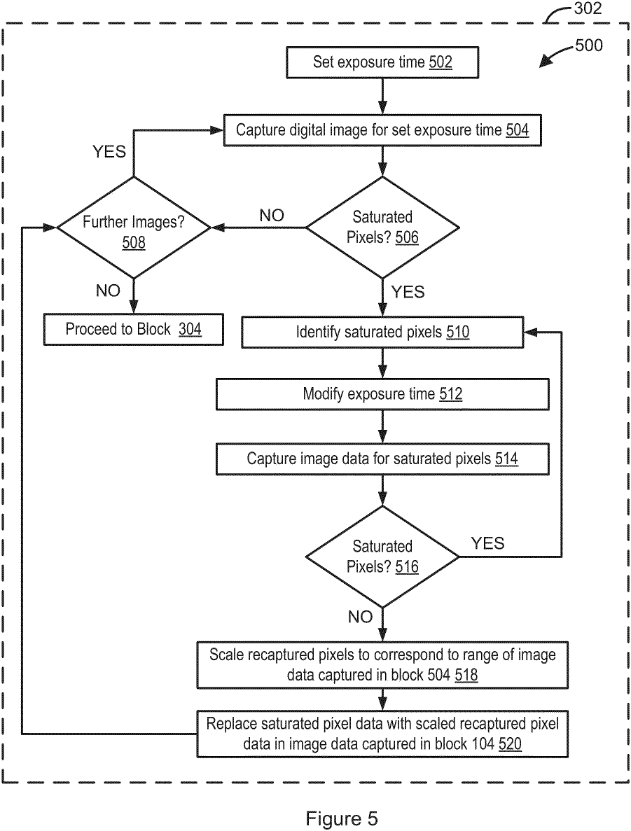

With reference now to FIG. 5, a flowchart illustrating one embodiment of a process 500 for another aspect of generating a series of images of a biological sample. The process 500 can be performed as a part of, or in the place of the step of block 302 of FIG. 3. In some embodiments, some or all of the steps of process 500 can be performed in addition to some or all of the steps of process 400. The process 500 begins at block 502, wherein an exposure time is set. In some embodiments, this exposure time can be a first exposure time. The exposure time can be, as discussed above, set by the computer 102 via one or several control signals sent to the photon resolving camera 104.

At decision step 504, one or several digital images are captured for the set, first exposure time. The one or several digital images can be captured by the photon resolving camera 104. The one or several digital can be send from the photon resolving camera 104 to the computer 102, which can evaluate the captured one or several images to determine if any of the pixels are saturated. If none of the pixels are saturated, then the process 500 proceeds to decision step 508, wherein it is determined if further images are to be captured. In some embodiments, the computer 102 can determine if further images are to be captured based on information relating to the number of images in the series of images already captured. If it is determined that further images are to be captured, then the process 500 proceeds to block 504 and proceeds as outlined above. Alternatively, if it is determined that no further images are to be captured, then the process 500 proceeds to block 304 of FIG. 3.

Returning again to decision step 506, if any of the pixels of the captured digital image are saturated, then the process 500 proceeds to block 510, wherein the saturated pixel(s) is identified. The saturated pixel(s) can be, in some embodiments, identified by the computer 102, can be identified by the photon resolving camera 104, and/or can be resolved by an integrated circuit such as a Field-programmable gate array included in the photon resolving camera 104 and/or between the photon resolving camera 104 and the computer 102. At block 512, the exposure time is modified. In some embodiments, the exposure time is modified to decrease the exposure time. In some embodiments, the exposure time is modified from the first exposure time to a second exposure time. In some embodiments, the second exposure time is less than the first exposure time, and modifying the exposure time can include decreasing the exposure time from the first exposure time to the second exposure time such that the second exposure time is less than the first exposure time. In some embodiments decreasing the exposure time can decrease the brightness level of the identified saturated pixels. The exposure time can be modified by the computer 102.

In some embodiments, and at block 512, it can be determined if the exposure time can be decreased. If the exposure time can be decreased, then modifying the exposure time can include decreasing the exposure time. In some embodiments, for example, the exposure time cannot be decreased as the exposure time may be limited by the amount of time required to read the imaging sensor in the photon resolving camera 104. In such an embodiment, instead of reading all of the pixels of the imaging sensor in the photon resolving camera 104, the read time can be decreased by decreasing the number of pixels of the imaging sensor that are read. In some embodiments, for example, only pixels previously identified as saturated are read, thereby decreasing the read time and enabling further decreases of the exposure time.

Thus, in some embodiments, upon detection of one or several saturated pixels, it can be determined if the exposure time can be decreased. If the exposure time cannot be decreased, then the number of pixels being read is decreased from the number of pixels read in the previously generated image. This can include limiting the number of pixels being read to only pixels identified in the previous image as saturated and/or limiting the number of pixels being read to a subset of the pixels identified as saturated in the previous image.

At block 514, image data at the modified exposure time is captured. In some embodiments, this can include capturing image data for some or all of the pixels in the image captured in block 504. Thus, in some embodiments, this can include capturing image data corresponding to the entirety of the image captured in block 504, and in some embodiments, this can include capturing image data corresponding to a portion of the image captured in block 504. In some embodiments, image data captured in block 514 can be for pixels identified as saturated. Thus, in some embodiments, image data for the at least one identified saturated pixel can be captured.

At block 516, it is determined if the image data captured in block 514 includes saturated pixels. This can include, for example, determining that the image data captured in block 514 does not include one or several saturated pixels, or includes one or several saturated pixels. If it includes saturated pixels, then the process 500 returns to block 510 and proceeds as outlined above.

Alternatively, if the image data captured in block 514 does not include saturated pixels, then the process 500 proceeds to block 518. At block 518, pixels in the image data captured in block 514 and corresponding to saturated pixels in block 514 are scaled. In some embodiments, this can include scaling the at least one pixel of image data captured in block 514 and corresponding to a saturated pixel. In some embodiments, this at least one pixel can be scaled based on the first exposure time and the second exposure time. This scaling can, covert the value of pixels in the image data captured at block 514 into the frame of reference of the image data captured in block 504. This scaling can include, for example, multiplying the value of the pixels in the image data captured at block 514 by the ratio of the second exposure time to the first exposure time. The pixels can be scaled by the computer 102.

At block 520, the saturated pixel data in the image data captured at block 504 is replaced by the scaled recaptured pixel data. In some embodiments, the saturated pixel data can be replaced by the computer 102. In other words, the pixel values for the saturated pixels from the image data captured at bock 504 are replaced by the corresponding pixels values for the scaled pixels from the image data captured in block 514. The modified image data from block 504 can be stored by the computer 102, and in some embodiments, can be stored by the computer 102 in the memory 104.

After the saturated pixel data is replaced, the process 500 proceeds to block 508, wherein it is determined if further images are to be captured. If it is determined that further images are to be captured, then the process 500 returns to block 504. Alternatively, if it is determined that further images are not to be captured, then the process 500 proceeds to block 304 of FIG. 3.

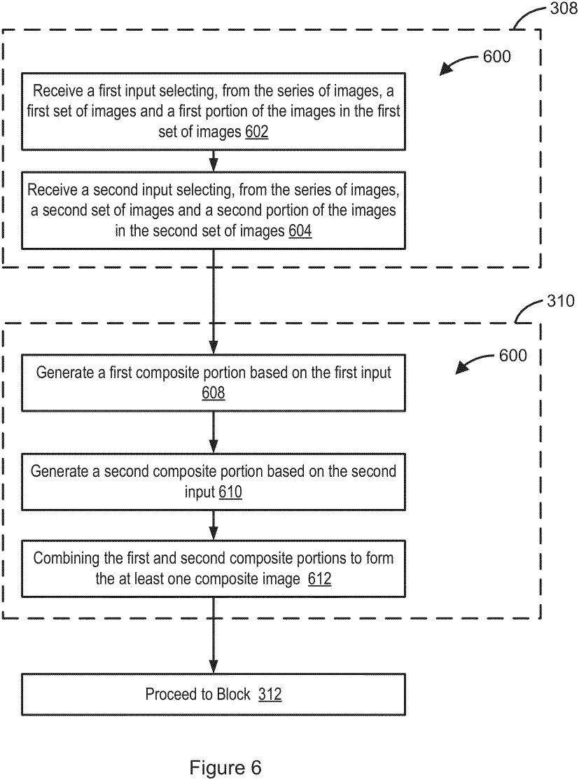

With reference now to FIG. 6, a flowchart illustrating one embodiment of a process 600 for generating a composite image of a biological sample is shown. The process 600 can be performed as a part of, or in the place of all or portions of the steps of blocks 308 and 310 of FIG. 3. The process 600 begins at block 602, wherein a first input selecting, from the series of images generated in block 302, a first set of images and a first portion of the images in the set of images is received. This first input can be received at the computer 102 via the I/O subsystem 206. At block 604, a second input selecting, from the series of images, a second set of images and a second portion of the images in the second set of images is received. In some embodiments, the first set of images and the second set of images can partially overlap in that they can each include some of the same images, and in some embodiments, the first set of images and the second set of images can be non-overlapping.

At block 608, a first composite portion is generated based on the first input. In some embodiments, this can include generating the first composite portion from the first portion of the images in the first set of images, and specifically from the first portion of each of the images in the first set of images. At block 610, a second composite portion can be generated based on the second input. In some embodiments, this can include generating the second composite portion from the second portion of the images in the second set of images. The first composite portion and the second composite portion can be generated by the computer 102.

At block 612, the first and second composite portions are combined to form at least one composite image. The first and second image portions can be combined by the computer 102. The composite image can, in some embodiments, be stored by the memory 204. After the first and second composite portions are combined to form the composite image, the process 600 proceeds to block 312 of FIG. 3.

This description should not be interpreted as implying any particular order or arrangement among or between various steps or elements except when the order of individual steps or arrangement of elements is explicitly described. Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

What is claimed is:

1. An imaging system comprising:
a sample plane configured to receive and hold a sample;
a photon resolving camera; and
a lens attached to the photon resolving camera, the photon resolving camera and the lens positioned to image the sample plane.

2. The imaging system of claim 1, further comprising a processor.

3. The imaging system of claim 2, wherein the photon resolving camera and the processor are configured for fluorescent and/or chemiluminescent imaging of a biological sample.

4. The imaging system of claim 2, wherein the photon resolving camera and the processor are configured for imaging of a western blot sample.

5. The imaging system of claim 2, wherein the sample comprises a fluorescent and/or chemiluminescent biological sample.

6. The imaging system of claim 5, wherein the sample comprises a western blot sample.

7. The imaging system of claim 2, wherein the processor is configured to generate a series of images of the sample plane.

8. The imaging system of claim 7, wherein the each of the series of images has the same exposure time.

9. The imaging system of claim 7, wherein at least some of the images in the series of images have different exposure times.

10. The imaging system of claim 7, wherein the processor is configured to generate a composite image from a selection of images in the series of images.

11. The imaging system of claim 10, wherein the processor is configured to generate and provide a live image stream displaying the composite image updated as a new image in the series of images is generated.

12. A method of fluorescent and/or chemiluminescent imaging of a biological sample, the method comprising:
generating a series of images of the biological sample with a photon resolving camera;
generating a composite image from at least some of the series of images; and
providing the composite image to a user.

13. The method of claim 12, further comprising:
providing the series of images to a user; and
receiving an input selecting at least some of the images in the series of images,
wherein the composite image is generated from the selected at least some of the images in the series of images.

14. The method of claim 13, wherein generating a series of images comprises:
setting an exposure time; and
capturing images at the set exposure time.

15. The method of claim 14, further comprising:
identifying a brightness level of at least one pixel of one of the images;
modifying the exposure time based on the brightness level to achieve a desired brightness level in a next captured image; and
capturing a next image at the modified exposure time.

16. The method of claim 15, wherein the at least one pixel comprises the brightest pixel in the image, and wherein modifying the exposure time to achieve a desired brightness level comprises increasing the exposure time to increase the brightness level of the brightest pixel in the image.

17. The method of claim 15, wherein the at least one pixel comprises the brightest pixel in the image, and wherein modifying the exposure time to achieve a desired brightness level comprises decreasing the exposure time from a first exposure time to a second exposure time to decrease the brightness level of the brightest pixel in the image.

18. The method of claim 15, wherein the exposure time is set to a first exposure time, the method further comprising:
identifying at least one pixel as saturated;
modifying the exposure time from the first exposure time to a second exposure time to decrease a brightness level of the saturated at least one pixel;
capturing image data at the modified exposure time of the at least one pixel;
determining that the at least one pixel is not saturated;
scaling the at least one pixel based on the second exposure time; and
replacing the saturated at least one pixel with the scaled at least one pixel.

19. The method of claim 18, wherein modifying the exposure time from the first exposure time to the second exposure time comprises decreasing the exposure time such that the second exposure time is less than the first exposure time.

20. The method of claim 19, when the at least one pixel is scaled based on both the first exposure time and the second exposure time.

21. The method of claim 12, wherein generating the composite image comprises:
receiving a first input selecting a first set of images and a first portion of each of the images in the first set of images;
receiving a second input selecting a second set of images and a second portion of each of the images in the second set of images;
generating a first composite portion from the first portion of each of the images in the first set of images;
generating a second composite portion from the second portion of each of the images in the second set of images; and
combining the first composite portion and the second composite portion.
